(19) United States
(12) Patent Application Publication
(10) Pub. No.: US 2018/0207219 A1
(43) Pub. Date: Jul. 26, 2018

(54) CHINESE MEDICINE HANGOVER CURING AGENT

(71) Applicants: Lei Wang, Beijing (CN); E Li, Beijing (CN)

(72) Inventors: Lei Wang, Beijing (CN); E Li, Beijing (CN)

(21) Appl. No.: 15/745,339

(22) PCT Filed: Aug. 31, 2015
(86) PCT No.: PCT/CN2015/088678

§ 371 Date: Jan. 16, 2018

(30) Foreign Application Priority Data
Aug. 10, 2015 (CN) 201510487918.8

(51) Int. Cl. A61K 36/734; A61K 36/62; A61K 36/605; A61K 36/288; A61K 36/899; A61K 36/888; A61K 36/752; A61P 39/02

(52) U.S. Cl. CPC A61K 36/734; A61K 36/62; A61K 36/605; A61P 39/02; A61K 36/899; A61K 36/888; A61K 36/752; A61K 36/288

(57) ABSTRACT

A Chinese medicine hangover curing agent. The agent is prepared from Chinese Hawthorn, Folium nelumbinis, Folium mori, Taraxaci herba, Lophatheri herba, common duckweed and tangerine leaves.

### TECHNICAL FIELD

The present invention relates to the technical fields of medicines, health products, food and the like, and in particular to a Chinese medicine alcoholic intoxication relieving agent.

### BACKGROUND ART

Alcoholic intoxication refers to a phenomenon that occurs when a person is out of control after drinking too much alcohol. Alcoholic intoxication can be generally divided into two types of severe alcoholic intoxication and mild alcoholic intoxication. There are the following symptoms for severe alcoholic intoxication: unsteady walking after drinking, allophasis, headache, vomiting and the like.

China is not only a country of long history of wine culture, but also a country of large production and consumption of wine beverages. With a significant improvement of material living standards of people in China, there has been an increase in the consumption of alcoholic (ethanol) beverages. In recent years, the incidence of adult alcoholism also increased accordingly. It is well-known that long-term alcohol drinking can cause systemic damage of multiple systems, and especially cause severe damages to gastrointestinal tracts and livers, and alcohol can directly or indirectly cause chronic gastritis, peptic ulcer and alcoholic cirrhosis. Acute alcoholism and alcohol dependence are one of the social problems today, and in case of unlimited excessive drinking or even alcoholism, in severe cases, a person will die from over-inhibition of the central nervous system by ethanol. Therefore, the alcoholism is harmful to the body.

### DISCLOSURE TO THE INVENTION

### Technical Problems

With the progress of the society, the development of science and technology and the improvement of people's living standards, people tend to have a lot of dinner parties for social entertainment, business contacts, family and friends and the like, and drinking is absolutely essential. High alcohol concentration in the body will cause alcoholic intoxication, alcoholism and even death. At present, there are many alcoholic intoxication relieving drugs available in the market. However, most of these alcoholic intoxication relieving drugs mainly focus on the protection of liver function while neglecting the damage caused by alcohol to other organs of the human body and fail to provide timely health care for other organs while maintaining the liver.

At present, there are many alcoholic intoxication relieving drugs in China, which mainly focus on traditional Chinese medicine or the combination of Chinese and Western medicines, but there are disadvantages such as high cost and unsatisfactory effects.

### Technical Solutions

In view of the above defects and problems in the prior art, an objective of the embodiment of the present invention is to provide an alcoholic intoxication relieving agent with better reliability. Under the guidance of Chinese medicine theory, the effects of synergistic prevention of alcoholic intoxication and alcoholic intoxication relieving are achieved by changing the process of ethanol absorption and metabolism, the symptoms of unstable walking after drinking, allophasis, headache and vomiting are alleviated, and the damage of alcohol to various organs of the body is reduced.

To achieve the above objective, the embodiments of the present invention adopt the following technical solution.

The Chinese medicine alcoholic intoxication relieving agent is characterized by being prepared from Fructus Crataegi, Folium Nelumbinis, Folium Mori, Herba Taraxaci, Herba Lophatheri, Herba Spirodelae, and Folium Citri tangerinae, wherein the Fructus Crataegi has effects of invigorating the spleen and promoting circulation of QI; the Folium Nelumbinis has effects of invigorating the spleen and ascending YIN; the Folium Mori has effects of clearing away lung-heat and moistening dryness; the Herba Lophatheri has effects of clearing away heat and relieving restlessness; the Herba Taraxaci has effects of clearing away heat and removing toxic substances; the Herba Spirodelae has effects of expelling pathogenic wind and promoting diuresis; and the Folium Citri tangerinae has effects of dispersing the stagnated liver-QI and promoting circulation of QI.

As a preferred selection from above technical solutions, the Chinese medicine alcoholic intoxication relieving agent is soaked in water with a single dosage for drinking: 10 g to 100 g of Fructus Crataegi, 1 g to 10 g of Folium Nelumbinis, 1 g to 10 g of Folium Mori, 1 g to 15 g of Herba Taraxaci, 1 g to 10 g of Herba Lophatheri, 1 g to 10 g of Herba Spirodelae and 1 g to 10 g of Folium Citri tangerinae.

As a preferred selection from above technical solutions, the Chinese medicine alcoholic intoxication relieving agent is prepared from the following components with 1 g by weight as one part: 9 parts of Fructus Crataegi, 1 part of Folium Nelumbinis, 3 parts of Folium Mori, 2 parts of Herba Taraxaci, 5 parts of Herba Lophatheri, 6 parts of Herba Spirodelae and 4 parts of Folium Citri tangerinae.

As a preferred selection from above technical solutions, the Chinese medicine alcoholic intoxication relieving agent is prepared in forms of decoctions, pills, capsules and oral liquids.

As a preferred selection from above technical solutions, the Chinese medicine alcoholic intoxication relieving agent is prepared by mixing extracts of Fructus Crataegi, Folium Nelumbinis, Folium Mori, Herba Taraxaci, Herba Lophatheri, Herba Spirodelae and Folium Citri tangerinae, i.e., alcoholic intoxication relieving enzymes according to a mass portion of 11:22:22:11:23:11.

As a preferred selection from above technical solutions, the Chinese medicine alcoholic intoxication relieving agent is taken 20 to 30 minutes before drinking wine.

According to the Chinese medicine alcoholic intoxication relieving agent provided by the embodiments of the present invention, in order to reduce the damage of alcohol to the body from the source, as compared with the traditional alcoholic intoxication relieving formula, the invention has effects of invigorating the spleen and stomach as well as preserving the liver, such that the liver cannot be damaged by alcoholism through the spleen and stomach. Through human feeding trails (see the following table), the peak concentration after acute alcohol intake can be reduced remarkably, the phenomenon of alcohol metabolism lag does not occur, and the metabolism of the organism to alcohol is accelerated.

Beneficial Effects of the Invention

The present invention has the following beneficial effects: with acceleration of alcohol metabolism, the alcohol concentration in blood can be reduced remarkably after heavy drinking, thereby reducing a variety of short-term discomforts after heavy drinking. The alcohol metabolism can be effectively accelerated to achieve the purpose of relieving alcoholic intoxication, and protecting people's kidneys, liver and other organs, and greatly reduce the damage of alcohol to the human body. The Chinese medicine alcoholic intoxication relieving agent disclosed by the present invention is low in economic cost and very simple in preparation at the same time. The Chinese medicine alcoholic intoxication relieving agent can be prepared into capsules, tablets, honeyed pills, oral liquids and other health products, medicines or food.

### EMBODIMENTS OF THE INVENTION

The technical solution of the present invention is described clearly and completely as below. It is apparent that the described embodiments are merely a part of embodiments of the present invention, rather than all of the embodiments. Based on the embodiments of the present invention, all of the other embodiments made by those ordinary skilled in the art without paying creative labor falls into the protection scope of the present invention.

### Embodiment 1

In an embodiment of the present invention, the Chinese medicine alcoholic intoxication relieving agent is prepared from Fructus Crataegi, Folium Nelumbinis, Folium Mori, Herba Taraxaci, Herba Lophatheri, Herba Spirodelae and Folium Citri tangerinae according to 10 g to 100 g of Fructus Crataegi, 1 g to 10 g of Folium Nelumbinis, 1 g to 10 g of Folium Mori, 1 g to 15 g of Herba Taraxaci, 1 g to 10 g of Herba Lophatheri, 1 g to 10 g of Herba Spirodelae and 1 g to 10 g of Folium Citri tangerinae. Sixteen volunteers are randomly divided into two groups of a traditional Chinese medicine group and a blank group. After the two groups are fasting for 4 hours, each volunteer in the Chinese medicine group drinks 100 ml of the Chinese medicine alcoholic intoxication relieving agent beverage of the embodiment of the present invention, and each volunteer in the blank group drinks 100 ml of normal saline; after drinking the Chinese medicine agent and the normal saline for 40 minutes, volunteers in the two groups drink 50 ml of 42-degreeErguotou; after 20 minutes of drinking, a breathalyzer is adopted to detect the alcohol content, and the results are as follows.

TABLE 1

| | Chinese | |
|---|---|---|
| | Medicine | Blank |
| Unit mg/100 ml | Group | Group |
| 1 | 14 | 44 |
| 2 | 12 | 42 |
| 3 | 15 | 45 |
| 4 | 9 | 40 |
| 5 | 17 | 50 |
| 6 | 10 | 47 |
| 7 | 11 | 49 |
| 9 | 13 | 43 |

The data results show that after each volunteer in the Chinese medicine group drinks 100 ml of traditional Chinese medicine alcoholic intoxication relieving agent and each volunteer in the blank group drinks 100 ml of normal saline, the alcohol concentration in the body of each volunteer in the Chinese medicine group is significantly lower than that in the body of each volunteer in the blank group.

The foregoing content is merely the specific embodiment of the present invention, however, the protection scope of the present invention is not limited thereto. Anyone skilled in the art may easily conceive changes and substitutions within the technical scope disclosed in the present invention, and all the changes and substitutions should be covered by the protection scope of the present invention. Therefore, the protection scope of the present invention should be subject to the protection scope of the claims.

What is claimed is:

1. A Chinese medicine alcoholic intoxication relieving agent, characterized by being prepared from Fructus Crataegi, Folium Nelumbinis, Folium Mori, Herba Taraxaci, Herba Lophatheri, Herba Spirodelae and Folium Citri tangerinae, wherein the Fructus Crataegi has effects of invigorating the spleen and promoting the circulation of QI;
the Folium Nelumbinis has effects of invigorating the spleen and ascending YIN; the Folium Mori has effects of clearing away lung-heat and moistening dryness; the Herba Lophatheri has effects of clearing away heat and relieving restlessness; the Herba Taraxaci has effects of clearing away heat and removing toxic substances; the Herba Spirodelae has effects of expelling pathogenic wind and promoting diuresis;
and the Folium Citri tangerinae has effects of dispersing the stagnated liver-QI and promoting circulation of QI.

2. The Chinese medicine alcoholic intoxication relieving agent, as a preferred selection from above technical solutions, being prepared from the following components with 1 g by weight as one part: 9 parts of Fructus Crataegi, 1 part of Folium Nelumbinis, 3 parts of Folium Mori, 2 parts of Herba Taraxaci, 5 parts of Herba Lophatheri, 6 parts of Herba Spirodelae and 4 parts of Folium Citri tangerinae;
the Chinese medicine alcoholic intoxication relieving agent according to claim 1 being characterized by being soaked in water with a single dosage for drinking: 10 g to 100 g of Fructus Crataegi, 1 g to 10 g of Folium Nelumbinis, 1 g to 10 g of Folium Mori, 1 g to 15 g of Herba Taraxaci, 1 g to 10 g of Herba Lophatheri, 1 g to 10 g of Herba Spirodelae and 1 g to 10 g of Folium Citri tangerinae.

3. The Chinese medicine alcoholic intoxication relieving agent, as a preferred selection from above technical solutions, being prepared by mixing extracts of Fructus Crataegi, Folium Nelumbinis, Folium Mori, Herba Taraxaci, Herba Lophatheri, Herba Spirodelae and Folium Citri tangerinae, i.e., alcoholic intoxication relieving enzymes according to a mass portion of 11:22:22:11:23:11.

4. The Chinese medicine alcoholic intoxication relieving agent according to claim 1, characterized in that the Chinese medicine alcoholic intoxication relieving agent is prepared in the orally-administrated forms, such as decoctions, pills, capsules and oral liquids.

5. The Chinese medicine alcoholic intoxication relieving agent according to claim 1, characterized in that the Chinese medicine alcoholic intoxication relieving agent is prepared by mixing extracts of Fructus Crataegi, Folium Nelumbinis, Folium Mori, Herba Taraxaci, Herba Lophatheri, Herba Spirodelae and Folium Citri tangerinae, i.e., alcoholic intoxication relieving enzymes according to 10 g to 100 g of Fructus Crataegi, 1 g to 10 g of Folium Nelumbinis, 1 g to 10 g of Folium Mori, 1 g to 15 g of Herba Taraxaci, 1 g to 10 g of Herba Lophatheri, 1 g to 10 g of Herba Spirodelae and 1 g to 10 g of Folium Citri tangerinae.

6. The Chinese medicine alcoholic intoxication relieving agent according to claim 1, characterized in that the Chinese medicine alcoholic intoxication relieving agent is taken 20 to 30 minutes before drinking wine.
